(19) United States
(12) Patent Application Publication
(10) Pub. No.: US 2010/0081939 A1
(43) Pub. Date: Apr. 1, 2010

(54) Technique of mapping sentinel lymph nodes using organic blue dyes combined with fluorescence imaging

(75) Inventor: Maoquan Chu, Shanghai (CN)

Correspondence Address: WARE FRESSOLA VAN DER SLUYS & ADOLPHSON, LLP, BRADFORD GREEN, BUILDING 5, 755 MAIN STREET, P O BOX 224, MONROE, CT, 06468, US

(73) Assignee: Shanghai Tongji University, Shanghai (CN)

(21) Appl. No.: 12/586,578

(22) Filed: Sep. 24, 2009

(30) Foreign Application Priority Data
Sep. 27, 2008 (CN) 200810200782.8

(51) Int. Cl. A61B 6/00

(52) U.S. Cl. 600/476

(57) ABSTRACT

This invention pertains to a combined technique using an organic blue dye and fluorescence imaging to map sentinel lymph nodes. The near-infrared fluorescence emitted by the blue dye is displayed by a fluorescent imaging system after being irradiated by an excitation light whose wavelength is no less than 570 nm. The blue dye is one of methylene blue, Evans blue, isosulfan blue, patent blue, or a combination of them. The invention solves the problem that a comparatively large incision on skin is inevitable when traditional method is used to detect tumors for sentinel lymph node mapping. The technique of this invention for mapping the sentinel lymph nodes eliminates the need for surgical management, since the sentinel lymph nodes in deep tissue are observed directly through the near-infrared fluorescence of the blue dye reducing pain of the patient. It is a non-radiation, convenient, fast, and accurate method with high clinical values.

### CROSS REFERENCE TO RELATED APPLICATIONS

The present application claims priority to Chinese Patent Application No. 200810200782.8, filed on Sep. 27, 2008. The Chinese patent application is hereby incorporated by reference in its entirety.

### TECHNICAL FIELD

This invention is in the biomedical field, involving the techniques of organic blue dyes combined with fluorescence imaging; especially a combination technique of organic blue dyes and the fluorescence imaging that methylene blue is mainly used as the stain to mark the sentinel lymph nodes.

### BACKGROUND OF THE INVENTION

The sentinel lymph nodes are the first group of lymph nodes reached by metastasizing cancer cells from a tumor. Chances are that cancer cells may have spread if metastasizing cancer cells appeared in the sentinel lymph nodes, and the extended surgery will be necessary, or vice versa. The status of the sentinel lymph nodes is the essential basis for the diagnosis of tumor spread and the establishment of therapeutic schemes. Compared with the traditional lymph node dissection, the sentinel lymph node biopsy technique is a great breakthrough in the tumor surgery that can avoid the unnecessary extended surgery and alleviate sufferings of patients.

The key technique of the sentinel lymph node biopsy is to accurately find the sentinel lymph node, namely the sentinel lymph node mapping. Up to now, blue dye staining, radiocolloid tracers, and a combination of both have been used for the sentinel lymph node mapping. The mainly used blue dye in clinic is the methylene blue. Evans blue, isosulfan blue and patent blue are also used in clinic. The methylene blue is nontoxic and non-radioactive. It has been used in clinic for over 100 years and proved to be save in practice. Since Morton et al (Morton DL, Wen DR, Wong JH et al. Technical details of intraoperative lymphatic mapping for early stage melanoma. Arch. Surg. 1992, 127:392-399) applied the blue dye in the sentinel lymph node biopsy of the melanoma, this technique has been widely used in the sentinel lymph node biopsy in clinic.

Sentinel lymph node mapping with blue dyes is performed by visual observation of the color (namely blue color) of the blue dyes. Generally speaking, the protocol of the technique is: the blue dye solution is subcutaneously injected into the patients or animals in the region close to the tumor, the skin is then incised, and the blue-dyed lymph nodes are observed directly by eyes. The practice indicates that only experienced doctors can find the sentinel lymph nodes by this technique. Moreover, patients or animals suffer more because of the large incision of the surgery.

Although the sentinel lymph nodes can be found by the radiation signal in the sentinel lymph node mapped with the radioactive nuclide tracing technology, the radiation would cause harm to patients and doctors. What's more, the cost is very high.

In 2004, Kim et al used near infrared fluorescent quantum dots (CdTe/CdSe) and in vivo fluorescent imaging technique for mapping the sentinel axillary lymph nodes in mice and pigs. They injected the quantum dots solution in pmol scale into the sole of mice and the legs of pigs. The sentinel axillary lymph nodes could be directly observed through the in vivo fluorescence emitted by the quantum dots. This technique is considered to be promising in clinic because it has a strong and stable fluorescence signal. However, the quantum dots contain toxic heavy metal ions such as Cd. The toxicity of Cd to the organism is just next to the hypertoxic Hg. Cd may cause several kinds of cancers in animals and human beings. International agency for research on cancer (IARC) has listed Cd into Class I carcinogens. Even covered with organic polymers or inorganic materials such as SiO₂, Cd ions may also leak from quantum dots and accumulate to be toxic in vivo.

### CONTENT OF INVENTION

This invention intends to solve the technical problem which is to provide a combined technique of organic blue dyes and fluorescence imaging to map sentinel lymph nodes.

This invention also intends to solve the technical problem of providing the application of the combined technique of organic blue dyes and fluorescence imaging.

The technical scheme taken by this invention when solving the technical problems mentioned above is this: a combined technique of organic blue dyes and fluorescence imaging to map sentinel lymph nodes. The fluorescence emitted from the organic blue dye, irradiated by the excitation light whose wavelength is no less than 570 nm, is displayed by a fluorescent imaging system. Where said organic blue dye is one of methylene blue, Evans blue, isosulfan blue or patent blue, which are clinically used as stain or indicator, or the combination of them.

During the reach for the organic blue dye which contains an excellent biocompatibility and dyeability against sentinel lymph nodes, the applicant of this invention found that such organic blue dye, which is extensive in clinical application, possesses fluorescence characteristics as well, nevertheless, it is too weak to attract sufficient attention. In clinical application, the sentinel lymph node can only be mapped by visual observation of the physical stain of methylene blue (blue color) after a surgical wound.

The applicant of this invention found that despite the low quantum yield of this organic blue dye, its fluorescence signal can be captured by high sensitive CCD camera, and displayed by imaging software. That is, the fluorescence emitted by organic blue dye can be imaged by the in vivo fluorescence imaging system. Therefore, the characteristic of organic blue dye being combined with the advanced fluorescence imaging technique can be used for tumor sentinel lymph nodes mapping.

Based on the scheme above, all the organic blue dye are dissolved in solvent which is de-ionized water, phosphate buffer solution or saline. The pH value of the solvent is between 6.8-7.5. The mass concentration of the solution is 0.5-2%.

Based on the scheme above, the wavelength of the excitation light mentioned above should be no less than 600 nm. Further optimization of the excitation light wavelength is 670-720 nm.

According to the application of the combined technique of organic blue dyes and fluorescence imaging mentioned above, the sentinel lymph nodes in vivo are first marked by organic blue dye, and then irradiated in vitro with the excitation light whose wavelength is no less than 570 nm from the fluorescence imaging system. The fluorescence emitted by organic blue dye is displayed on the fluorescence imaging system. In this process, the organic blue dye mentioned is one of methylene blue, Evans blue, isosulfan blue or patent blue, or the combination of them. Among them, methylene blue is the optimal selection.

In detail, by subcutaneous injection around the animal tumor, the organic blue dye flows into sentinel lymph node and marks it, and then irradiate in vitro with the excitation light whose wavelength is no less than 570 nm to make the organic blue dye in sentinel lymph nodes around the animal tumor emit near-infrared fluorescence which forms the image of animal tissue and organs. The process and output of the result that displays the vivo sentinel lymph nodes is accomplished by the fluorescence imaging system.

Where the organism with lymphatic system refers to human beings, mammalian, reptilian, and birds.

The technique of this invention can be used for mapping the sentinel lymph nodes of normal animal, that is, the irradiation in vitro, after the subcutaneous injection of organic blue dye in normal animal, with the excitation light whose wavelength is no less than 570 nm form the fluorescence imaging system makes the organic blue dye in sentinel lymph nodes around the injection position emit near-infrared fluorescence which is displayed by the in vivo fluorescent imaging system.

The advantages of this invention are as follows:

It solves the problem that a comparatively large incision on skin is inevitable when the traditional method is used for tumor sentinel lymph node mapping. Using the technique of this invention for mapping the sentinel lymph nodes would eliminate the needs for surgical management. This technique can reduce the pain of the patient, and in the mean time, can avoid the radioactive harms and the toxicity of heavy metals when radiocolloid and quantum dots are used for the sentinel lymph node mapping, respectively. This invention uses organic blue dye with excellent biocompatibility such as methylene blue, associated with the fluorescent imaging technique, to display the fluorescence of organic blue dye, so as to observe by naked eye, on fluorescence imaging system after being excited by a light whose wavelength is no less than 570 nm. It is a convenient, fast, and accurate method with highly clinical value.

### DESCRIPTION OF FIGURES

The patent or application file contains at least one drawing executed in color. Copies of this patent or patent application publication with color drawing(s) will be provided by the Office upon request and payment of the necessary fee.

FIG. 1: brightfield and fluorescent photographs of the sentinel lymph nodes of a rabbit without being mapped with organic blue dyes.

FIG. 2: brightfield and fluorescent photographs of the sentinel lymph nodes of a rabbit mapped with organic blue dyes.

### DETAILED IMPLEMENTATION

### Example

A combined technique of organic blue dyes mapping the sentinel lymph nodes and fluorescence imaging. With the irradiation of 671˜705 nm wavelength excitation light in the fluorescence imaging system, the fluorescence of the organic blue dye can be shown on the fluorescence imaging system. The mentioned organic blue dye is methylene blue.

Phosphate buffer solutions (pH=7.2) dissolved methylene blue with mass concentration of 0.2%, 0.5%, 1%, 2% were prepared, respectively. Methylene blue solutions with different concentration (0.8 ml) were subcutaneously injected into male New Zealand white rabbits (n=4) in the region surrounding the mammary areola of the second pair of breasts. The rabbits were then irradiated using an excitation light with 671˜705 nm wavelength. The emission filter was 750 nm. Inspect the fluorescence signal from the second pair of breasts to the axillary using the in vivo imaging system.

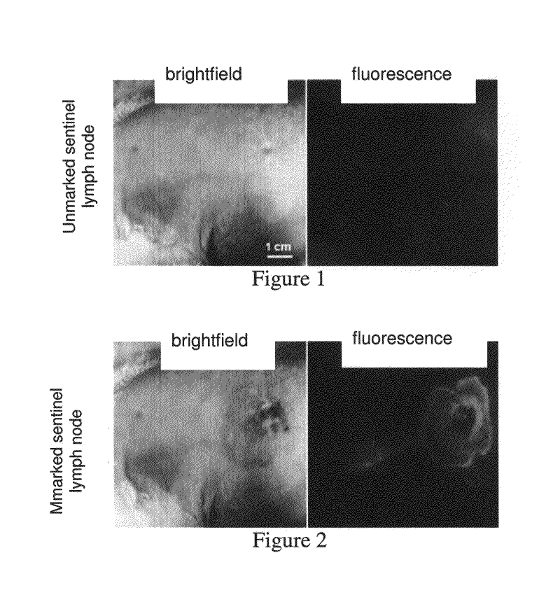

Refer to FIG. 1 (The photographs of the rabbit without the sentinel lymph nodes mapped by the organic blue dye) and FIG. 2 (The photographs of the rabbit with the sentinel lymph nodes mapped by the organic blue dye). We can find that, without the need of cutting the skin of the rabbit, the fluorescence of methylene blue flows from the second pair of breasts to the axillary on the same side in about 1 minute. And 1˜2 fluorescence spots with diameter of 0.2˜0.5 cm are found in the axillary. When the concentration of the injected methylene blue solution is 0.2%, the fluorescence spots in the axillary can keep for 1-2 hours. The maintaining time of the fluorescence signal from the spots prolongs with the increasing concentration of the methylene blue solution. The fluorescence signal from the spots can maintain for at most 24 hours when the concentration of injected methylene blue solution is 2%.

Through the traditional method of the sentinel lymph nodes mapping (skin incision from breasts to the axillary), it is found that the fluorescent spots are the sentinel lymph nodes.

The fluorescence imaging system applied in this application example is the CRI Maestro™ in vivo imaging system of the CRI Corporation in USA.

The example can be also performed using the Photon Imagor In Vivo Fluorescence Imaging System of BioSpace Corporation in France, In Vivo Fluorescence Imaging System of Kodak in USA, or in the products of Xenogen in USA.

What is claimed is:

1. A combined technique of organic blue dye and fluorescence imaging for mapping sentinel lymph nodes, comprising:
irradiating an organic blue dye by an excitation light whose wavelength is no less than 570 nm to cause a fluorescence emission from the organic blue dye; and
displaying an image formed by the fluorescence on a fluorescence imaging system,
wherein the organic blue dye is one of methylene blue, Evans blue, isosulfan blue or patent blue, which are clinically used as stain or indicator, or a combination of said dyes.

2. The combined technique of organic blue dye and fluorescence imaging for mapping sentinel lymph nodes according to claim 1, wherein the organic blue dye is dissolved in a solvent which is de-ionized water, phosphate buffer solution or saline, and wherein the pH value of the solvent is between 6.8 and 7.5 and the blue dye concentration in the solution is 0.5-2%.

3. The combined technique of organic blue dye and fluorescence imaging for mapping sentinel lymph nodes according to claim 1, wherein the wavelength of the excitation light is no less than 600 nm.

4. An application of the combined technique of organic blue dye and fluorescence imaging for mapping sentinel lymph nodes according to claim 1, comprising:
marking the sentinel lymph nodes of a living object (living animal or human) by the organic blue dye,
irradiating the living object externally by the excitation light whose wavelength is no less than 570 nm form the fluorescence imaging system to cause a florescence emission from the living object; and
displaying a near-infrared fluorescence image of the marked sentinel lymph nodes on the fluorescence imaging system,
wherein the organic blue dye is one of methylene blue, Evans blue, isosulfan blue or patent blue, which are clinically used as stain or indicator, or a combination of said dyes.

5. The combined technique of organic blue dye and fluorescence imaging for mapping sentinel lymph nodes according to claim 2, wherein the wavelength of the excitation light is no less than 600 nm.
